(19) United States
(12) Patent Application Publication
(10) Pub. No.: US 2019/0299029 A1
(43) Pub. Date: Oct. 3, 2019

(54) CHARGED PARTICLE BEAM TREATMENT APPARATUS

(71) Applicant: SUMITOMO HEAVY INDUSTRIES, LTD., Tokyo (JP)

(72) Inventor: Junichi Inoue, Ehime (JP)

(21) Appl. No.: 16/360,844

(22) Filed: Mar. 21, 2019

(30) Foreign Application Priority Data
Mar. 29, 2018 (JP) 2018-065175

(51) Int. Cl. A61N 5/10; A61B 6/03; A61B 6/00

(52) U.S. Cl. CPC A61N 5/1049; A61B 6/032; A61N 2005/1061; A61B 6/5211; A61N 5/1081

(57) ABSTRACT

A charged particle beam treatment apparatus includes an irradiation unit that irradiates a patient with a charged particle beam, a lesion area position detection unit that detects a position of a lesion area of the patient, and a control unit that controls the irradiation unit, based on the position of the lesion area detected by the lesion area position detection unit. The control unit adjusts an irradiation position of the charged particle beam in a plane direction orthogonal to an irradiation axis of the charged particle beam so as to track variations in the position of the lesion area in the plane direction. When the position of the lesion area in a depth direction along the irradiation axis is out of a predetermined range, the control unit stops irradiation, and when the position of the lesion area falls again within the predetermined range, the control unit resumes the irradiation.

### RELATED APPLICATIONS

Priority is claimed to Japanese Patent Application No. 2018-065175, filed Mar. 29, 2018, the entire content of which is incorporated herein by reference.

### BACKGROUND

### Technical Field

Certain embodiments of the present invention relate to a charged particle beam treatment apparatus.

### Description of Related Art

In the related art, a treatment apparatus is known as a technique in a field of a charged particle beam treatment apparatus. The treatment apparatus performs so-called moving body tracking treatment for irradiating a lesion area of a patient with a radioactive ray so as to track a motion of the lesion area of the patient.

### SUMMARY

It is desirable to provide a charged particle beam treatment apparatus which can improve accuracy in irradiating a lesion area with a charged particle beam.

According to an embodiment of the present invention, there is provided a charged particle beam treatment apparatus including an irradiation unit that irradiates a patient with a charged particle beam, a lesion area position detection unit that detects a position of a lesion area of the patient, and a control unit that controls the irradiation unit, based on the position of the lesion area detected by the lesion area position detection unit. The control unit adj usts an irradiation position of the charged particle beam in a plane direction orthogonal to an irradiation axis of the charged particle beam so as to track variations in the position of the lesion area in the plane direction. In a case where the position of the lesion area in a depth direction along the irradiation axis is out of a predetermined range, the control unit stops irradiation using the charged particle beam, and in a case where the position of the lesion area falls again within the predetermined range, the control unit resumes the irradiation using the charged particle beam.

### BRIEF DESCRIPTION OF THE DRAWINGS

FIG. 1 a view illustrating a charged particle beam treatment apparatus according to an embodiment.

FIG. 2 is an enlarged view illustrating an irradiation unit of the charged particle beam treatment apparatus according to the embodiment.

FIG. 3 is a view including a CT scanner of the chargedparticle beam treatment apparatus according to the embodiment.

FIGS. 4A and 4B are views illustrating a layer set for a tumor.

FIG. 5 is a flowchart illustrating a procedure of charged particle beam treatment performed by the charged particle beam treatment apparatus.

FIG. 6A is a view illustrating an estimated tumor position of a treatment plan map, and FIG. 6B is a view illustrating a tumor measured position measured by the CT scanner.

### DETAILED DESCRIPTION

Here, in a case of performing treatment by using a charged particle beam, a position in a depth direction (direction along an irradiation axis) of the lesion area at an irradiation position is adjusted by adjusting energy of the charged particle beam so as to change a range of the charged particle beam. That is, in a case of performing moving body tracking treatment by using the charged particle beam, it becomes necessary to change the range of the charged particle beam when the lesion area varies in the depth direction. It is difficult to instantaneously adjust the energy of the charged particle beam. In some cases, the range of the charged particle beam cannot sufficiently track variations in the lesion area in the depth direction. For example, when the range of the charged particle beam completely varies, in some cases, the lesion area may further move to a different position in the depth direction. In this case, an unexpected location is irradiated with the charged particle beam. Consequently, accuracy in irradiating the lesion area with the charged particle beam becomes poor.

The irradiation position of the charged particle beam in the plane direction can be promptly adjusted. Therefore, the control unit can promptly adjust the irradiation position of the charged particle beam in the plane direction so as to track variations in the position of the lesion area in the plane direction. In this manner, the irradiation position of the charged particle beam can satisfactorily track the variations in the position of the lesion area in the plane direction. On the other hand, in order to adjust the irradiation position of the charged particle beam in a depth direction, it is necessary to change energy of the charged particle beam. Accordingly, it requires time to adjust the irradiation position of the charged particle beam. Therefore, in a case where the position of the tumor in the depth direction along the irradiation axis is out of the predetermined range, the control unit stops the irradiation using the charged particle beam, and if the position of the tumor falls again within the predetermined range, the control unit resumes the irradiation using the charged particle beam. That is, when the position of the lesion area in the depth direction less deviates and a planned location can be irradiated with the charged particle beam, the control unit performs the irradiation using the charged particle beam. When the position of the lesion area in the depth direction greatly deviates, the control unit stops the irradiation so that an unplanned location is not irradiated with the charged particle beam. In this manner, the irradiation unit can prevent the unplanned location from being irradiated with the charged particle beam. According to the above-described configuration, it is possible to improve accuracy in irradiating the lesion area with the charged particle beam.

The lesion area position detection unit may include a CT scanner which acquires a CT image of the lesion area. In this case, the lesion area position detection unit can accurately detect the position of the lesion area.

The lesion area position detection unit may detect a body surface motion of the patient, and may detect the position of the lesion area by estimating the position of the lesion area from the body surface motion. In this case, the lesion area position detection unit can detect the position of the lesion area without using a large-scale device such as a CT scanner.

According to the present invention, it is possible to provide a charged particle beam treatment apparatus which can improve accuracy in irradiating a lesion area with a charged particle beam.

Hereinafter, charged particle beam treatment apparatus according to an embodiment of the present invention will be described with reference to the accompanying drawings. In describing the drawings, the same reference numerals will be given to the same elements, and repeated description will be omitted.

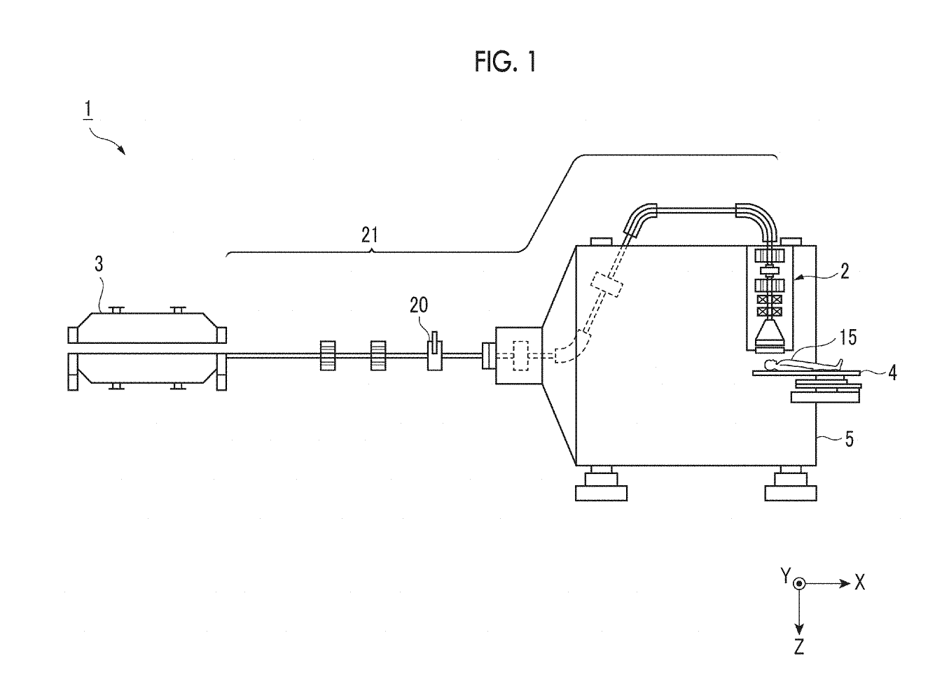

As illustrated in FIG. 1, a charged particle beam treatment apparatus 1 according to an embodiment of the present invention is a device used for cancer treatment performed using radiation treatment, and includes an accelerator 3 that accelerates a charged particle generated by an ion source (not illustrated) so as to emit the charged particle as a charged particle beam, an irradiation unit 2 that irradiates an irradiation target body with the charged particle beam, and a beam transport line 21 that transports the charged particle beam emitted from the accelerator 3 to the irradiation unit 2. The irradiation unit 2 is attached to a rotary gantry 5 disposed so as to surround a treatment table 4. The irradiation unit 2 is rotatable around the treatment table 4 by using the rotary gantry 5. A patient 15 who is a subject of charged particle beam treatment is placed on the treatment table 4.

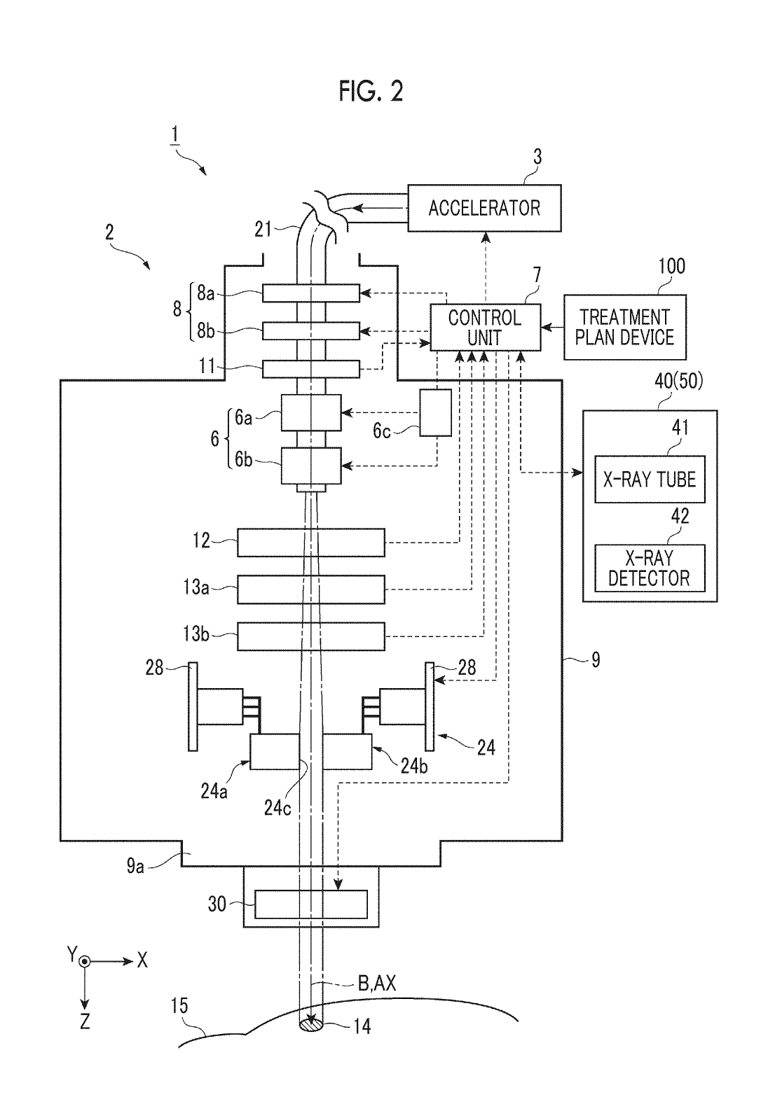

FIG. 2 is a schematic configuration diagram in the vicinity of the irradiation unit 2 of the charged particle beam treatment apparatus in FIG. 1. In the following description, directions will be described using terms of an “X-axis direction”, a “Y-axis direction”, and a “Z-axis direction”. The “Z-axis direction” is a direction in which a base axis AX of a charged particle beam B extends, and is a depth direction of irradiation of the charged particle beam B. The “base axis AX” is set as an irradiation axis of the charged particle beam B in a case where the charged particle beam B is not deflected by a scanning electromagnet 6 (to be described later). FIG. 2 illustrates a state where the irradiation target body is irradiated with the charged particle beam B along the base axis AX. The “X-axis direction” is one direction within a plane orthogonal to the Z-axis direction. The “Y-axis direction” is a direction orthogonal to the X-axis direction within the plane orthogonal to the Z-axis direction.

First, referring to FIG. 2, a schematic configuration of the charged particle beam treatment apparatus 1 according to the present embodiment will be described. The charged particle beam treatment apparatus 1 is an irradiation apparatus adopting a scanning method. The scanning method is not particularly limited, and line scanning, raster scanning, or spot scanning may be adopted. As illustrated in FIG. 2, the charged particle beam treatment apparatus 1 includes the accelerator 3, the irradiation unit 2, the beam transport line 21, and a control unit 7.

The accelerator 3 is a device that accelerates the charged particle so as to emit the charged particle beam B having preset energy. Examples of the accelerator 3 include a cyclotron, a synchrotron, a synchrocyclotron, and a linear accelerator. In a case where the cyclotron which emits the charged particle beam B having the preset energy is adopted as the accelerator 3, an energy adjustment unit 20 (refer to FIG. 1) is adopted so as to be capable of adjusting (lowering) the energy of the charged particle beam to be fed to the irradiation unit 2. The synchrotron can easily change the energy of the charged particle beam to be emitted. Accordingly, in a case where the synchrotron is adopted as the accelerator 3, the energy adjustment unit 20 may be omitted. The accelerator 3 is connected to the control unit 7 so that a current to be supplied is controlled. The charged particle beam B generated by the accelerator 3 is transported to an irradiation nozzle 9 by the beam transport line 21. The beam transport line 21 connects the accelerator 3, the energy adjustment unit 20, and the irradiation unit 2 to each other, and transports the charged particle beam emitted from the accelerator 3 to the irradiation unit 2.

The irradiation unit 2 irradiates a tumor (lesion area) 14 inside a body of a patient 15 with the charged particle beam B. The charged particle beam B is obtained by accelerating a particle having an electric charge at high speed, and examples thereof include a proton beam, a heavy particle (heavy ion) ray, and an electron beam. Specifically, the irradiation unit 2 irradiates the tumor 14 with the charged particle beam B emitted from the accelerator 3 which accelerates the charged particle generated by the ion source (not illustrated) and transported by the beam transport line 21. The irradiation unit 2 includes a scanning electromagnet 6, a quadrupole electromagnet 8, a profile monitor 11, a dose monitor 12, position monitors 13a and 13b, a multi-leaf collimator 24, and a degrader 30. The scanning electromagnet 6, the respective monitors 11, 12, 13a, and 13b, the quadrupole electromagnet 8, and the degrader 30 are accommodated in an irradiation nozzle 9. In this way, the irradiation unit 2 is configured to include the irradiation nozzle 9 in which each main configuration element is accommodated in an accommodation body. The quadrupole electromagnet 8, the profile monitor 11, the dose monitor 12, the position monitors 13a and 13b, and the degrader 30 may be omitted.

The scanning electromagnet 6 includes an X-axis direction scanning electromagnet 6a and a Y-axis direction scanning electromagnet 6b. The X-axis direction scanning electromagnet 6a and the Y-axis direction scanning electromagnet 6b are respectively configured to include a pair of electromagnets, and change a magnetic field between the pair of electromagnets in accordance with a current to be supplied from the control unit 7 so as to perform scanning using the charged particle beam B passing between the electromagnets. The X-axis direction scanning electromagnet 6a performs the scanning using the charged particle beam B in an X-axis direction, and the Y-axis direction scanning electromagnet 6b performs the scanning using the charged particle beam B in a Y-axis direction. The scanning electromagnets 6 are arranged on the base axis AX, and in this order on a downstream side of the charged particle beam B from the accelerator 3.

The quadrupole electromagnet 8 includes an X-axis direction quadrupole electromagnet 8a and a Y-axis direction quadrupole electromagnet 8b. The X-axis direction quadrupole electromagnet 8a and the Y-axis direction quadrupole electromagnet 8b narrow the charged particle beam B so as to converge in accordance with the current supplied from the control unit 7. The X-axis direction quadrupole electromagnet 8a causes the charged particle beam B to converge in the X-axis direction, and the Y-axis direction quadrupole electromagnet 8b causes the charged particle beam B to converge in the Y-axis direction. A beam size of the charged particle beam B can be changed by changing the current to be supplied to the quadrupole electromagnet 8 so as to change a narrowing amount (convergence amount). The quadrupole electromagnet 8 is located on the base axis AX in this order between the accelerator 3 and the scanning electromagnet 6. The beam size is a size of the charged particle beam B in an XY-plane. A beam shape is a shape of the charged particle beam B in the XY-plane.

The profile monitor 11 detects the beam shape and the position of the charged particle beam B for alignment during initial setting. The profile monitor 11 is located on the base axis AX and between the quadrupole electromagnet 8 and the scanning electromagnet 6. The dose monitor 12 detects a dose of the charged particle beam B. The dose monitor 12 is located on the base axis AX and on the downstream side from the scanning electromagnet 6. The position monitors 13a and 13b detect and monitor the beam shape and position of the charged particle beam B. The position monitors 13a and 13b are located on the base axis AX and on the downstream side of the charged particle beam B from the dose monitor 12. The respective monitors 11, 12, 13a, and 13b output a detection result to the control unit 7.

The degrader 30 lowers the energy of the chargedparticle beam B passing therethrough, and performs fine tuning on the energy of the charged particle beam B. According to the present embodiment, the degrader 30 is provided in a tip portion 9a of the irradiation nozzle 9. The tip portion 9a of the irradiation nozzle 9 is an end portion on the downstream side of the charged particle beam B.

Based on a signal output from the control unit 7, the multi-leaf collimator 24 defines an irradiation field 60 of the charged particle beam B in the plane direction perpendicular to a direction of the irradiation axis, and has a charged particle beam blocking portions 24a and 24b including a plurality of comb teeth. The charged particle beam blocking portions 24a and 24b are arranged so as to match each other, and an opening portion 24c is formed between the charged particle beam blocking portions 24a and 24b. The opening portion 24c defines the irradiation field of the charged particle beam B. The multi-leaf collimator 24 blocks a portion of the charged particle beam B which is used in irradiating a peripheral edge portion of the irradiation field by allowing the charged particle beam B to pass through the opening portion 24c. When the irradiation using the charged particle beam is performed by means of a scanning method, the irradiation field of the charged particle beam B is defined by a route where the scanning is performed using the charged particle beam and the irradiation is performed using the charged particle beam. In this case, the charged particle beam is blocked in an end portion of the irradiation field by using the multi-leaf collimator 24. Accordingly, a penumbra (no more dose distribution) is improved.

Further, the multi-leaf collimator 24 can change the position and shape of the opening portion 24c, that is, the irradiation field by moving the charged particle beam blocking portions 24a and 24b forward and rearward in a direction orthogonal to the Z-axis direction. Furthermore, the multi-leaf collimator 24 is guided along the direction of the irradiation axis by a linear guide 28, and is movable along the Z-axis direction. The multi-leaf collimator 24 is located on the downstream side of the position monitor 13b.

For example, the control unit 7 is configured to include a CPU, a ROM, and a RAM. Based on a detection result output from the respective monitors 11, 12, 13a, and 13b, the control unit 7 controls the accelerator 3, the scanning electromagnet 6, the quadrupole electromagnet 8, and the multi-leaf collimator 24.

The control unit 7 of the charged particle beam treatment apparatus 1 is connected to a treatment plan device 100 which performs treatment planning of the charged particle beam treatment. The treatment plan device 100 measures the tumor 14 of the patient 15 by using CT before the treatment is performed, and plans dose distribution (dose distribution of the charged particle beam to be used in the irradiation) at each position of the tumor 14. Specifically, the treatment plan device 100 prepares a treatment plan map (treatment plan information) for the tumor 14. The treatment plan device 100 transmits the prepared treatment plan map to the control unit 7.

In a case where the irradiation using the charged particle beam is performed by means of the scanning method, the tumor 14 is virtually divided into a plurality of layers in the Z-axis direction, and the irradiation is performed by scanning one layer of the tumor 14 with the charged particle beam so as to follow a scanning route (scanning pattern) determined in a treatment plan. After the one layer is completely irradiated with the charged particle beam, a subsequent layer adjacent thereto is irradiated with the charged particle beam B. In this way, every layered region divided in the Z-axis direction is repeatedly irradiated one by one with the charged particle beam B. In this manner, the whole three-dimensional tumor 14 is irradiated irradiation with the charged particle beam B.

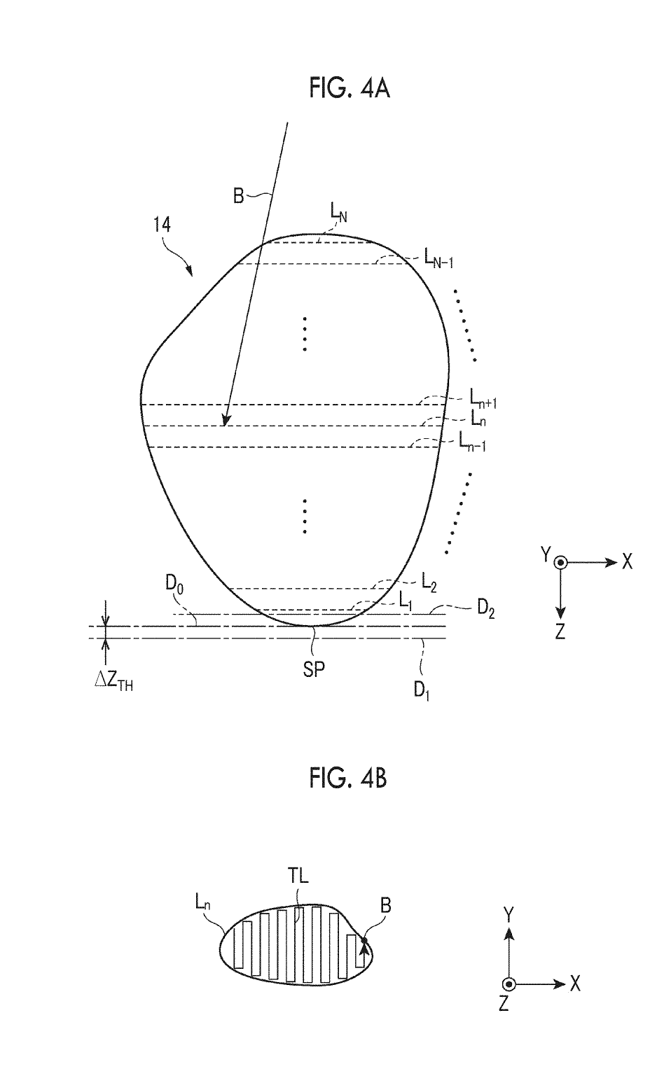

An irradiation image of charged particle beam of the scanning electromagnet 6 in accordance with the control of the control unit 7 will be described with reference to FIGS. 4A and 4B. FIG. 4A illustrates the tumor 14 virtually sliced into a pluralityof layers in the depth direction, and FIG. 4B illustrates a scanning image of the charged particle beam in one layer when viewed in the depth direction, respectively.

As illustrated in FIG. 4A, the tumor 14 is virtually sliced into a plurality of layers in the depth direction of the irradiation. In this example, deeper (longer range of the charged particle beam B) layers are sequentially and virtually sliced into a layer L₁, a layer L₂, . . . a layer _{Ln-1}, a layer _{Ln}, a layer _{Ln+1}, . . . a layer _{LN-1}, a layer _{LN}, and a layer _{N}. As illustrated in FIG. 4B, while the charged particle beam B draws a beam trajectory TL, a plurality of irradiation spots of the layer Ln is irradiated with the charged particle beam B. That is, the irradiation nozzle 9 controlled by the control unit 7 moves on the beam trajectory TL.

The above-described treatment plan map includes information on a position of the tumor 14 (hereinafter, referred to as an “estimated tumor position”) estimated to be positioned on the treatment table 4. In addition, the treatment plan map includes information on a scanning route of the charged particle beam B for the estimated tumor position. The control unit 7 reads out the estimated tumor position and the scanning route which are determined in the treatment plan map. In principle, the control unit 7 sets the estimated tumor position as a planned irradiation position, and controls the irradiation unit 2 so that the planned irradiation position is irradiated with the charged particle beam in accordance with the scanning route. Therefore, in principle, if the tumor 14 of the patient 15 on the treatment table 4 does not vary from the estimated tumor position, the tumor 14 is scanned and irradiated with the charged particle beam B in accordance with the estimated tumor position and the scanning route.

The “estimated tumor position” and the “planned irradiation position” which are described above, and an “actually measured tumor position” to be described later are all concepts including a three-dimensional shape or a three-dimensional position of the tumor 14 (position in a translational direction of three X, Y, and Z axes, and position in a rotation direction around the three X, Y, and Z axes). The variations in the position of the tumor 14 within the XY-plane correspond to the variations in the “plane direction” orthogonal to the irradiation axis (base axis AX) of the charged particle beam B. In addition, the variations in the position of the tumor 14 in the Z-axis direction correspond to the variations in the “depth direction” along the irradiation axis.

Furthermore, as illustrated in FIG. 2, the charged particle beam treatment apparatus 1 includes a lesion area position detection unit 50 that detects a position of the tumor 14 of the patient 15 on the treatment table 4 during the irradiation of the charged particle beam B. As a position measuring unit configured in this way, for example, an X-ray CT scanner or digital radiography (DR) may be adopted. In the following description, an example will be described where the charged particle beam treatment apparatus 1 includes a CT scanner 40 as the lesion area position detection unit 50.

The CT scanner 40 is a type called a cone beam CT scanner (CBCT scanner), and is used in order to accurately recognize the position of the tumor 14 on the treatment table 4 with respect to the irradiation unit 2. Specifically, prior to the charged particle beam treatment, a tomographic image (CT image) of the patient 15 is prepared using the CT scanner 40 in a state where the CT scanner 40 is set on the treatment table 4. Based on the CT image, the position of the tumor 14 of the patient 15 is recognized.

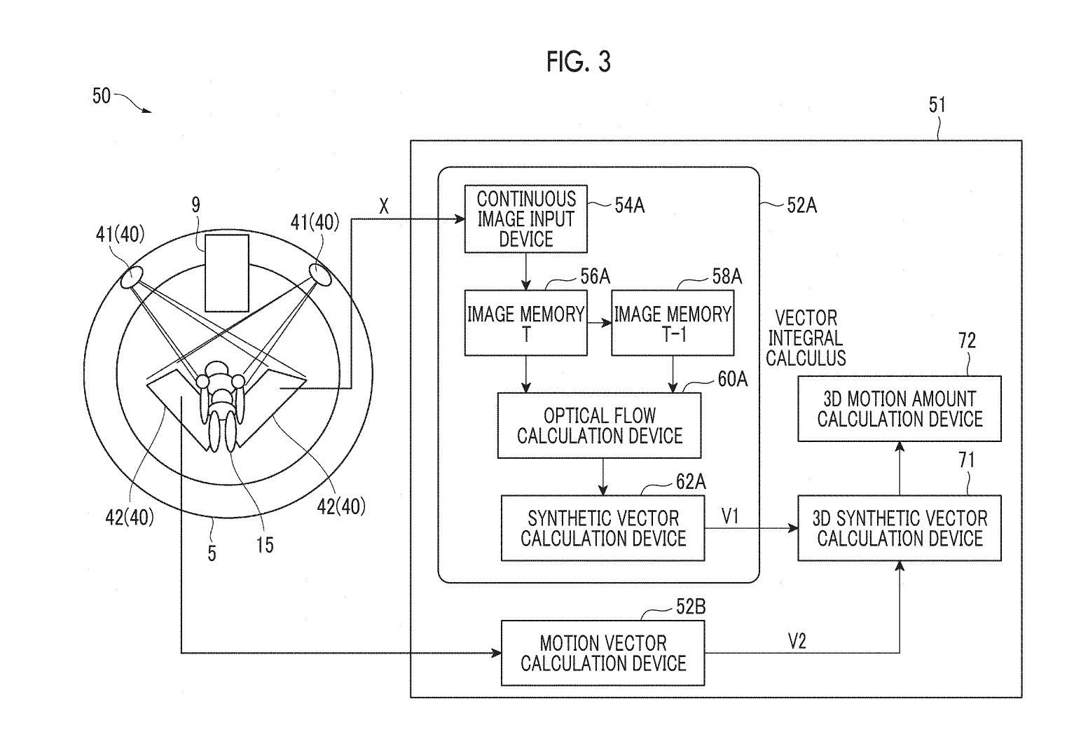

As also illustrated in FIG. 3, the CT scanner 40 includes an X-ray tube 41 which irradiates the patient 15 with an X-ray. Every X-ray tube 41 is installed on both sides of the irradiation nozzle 9. The CT scanner 40 includes two X-ray detectors 42 for respectively detecting the X-ray from each of the X-ray tubes 41. A set of the X-ray tube 41 and the X-ray detector 42 is located at mutually opposite positions across the treatment table 4. The X-ray tube 41 and the X-ray detector 42 are supported by the above-described rotary gantry 5, and are configured to be rotatable. Both of these are integrally rotated around the treatment table 4. The X-ray is emitted from the X-ray tube 41, and the X-ray passing through the patient 15 on the treatment table 4 is detected by the X-ray detector 42. The X-ray detector 42 acquires X-ray image data of the patient 15. The lesion area position detection unit 50 includes a 3D lesion area tracking device 51. The 3D lesion area tracking device 51 is incorporated in the control unit 7. The X-ray image data is transmitted to the 3D lesion area tracking device 51 of the control unit 70. The control unit 7 performs image reconstruction processing by means of predetermined calculation, based on the above-described X-ray image data, and generates the CT image inside the patient 15. Based on the CT image, the control unit 7 acquires an actual position of the tumor 14 of the patient 15 on the treatment table 4.

The 3D lesion area tracking device 51 is configured to include motion vector calculation devices 52A and 52B disposed in each imaging direction so as to calculate motion vector by calculating an instantaneous optical flow from a real-time image input from the respective X-ray detectors 42, a three-dimensional (3D) synthetic vector calculation device 71 which measures a three-dimensional motion vector from a two-directional motion vector obtained by the motion vector calculation devices 52A and 52B, and a 3D movement amount calculation device 72 which calculates a three-dimensional movement amount by performing vector integral calculus on an output of the 3D synthetic vector calculation device 71.

The motion vector calculation devices 52A and 52B are configured to include a continuous image input device 54A for inputting a continuous image from the X-ray detector 42, a current image memory 56A for storing the image input from the continuous image input device 54 as a current image, a previous image memory 58A for storing the image temporarily stored in the current image memory 56A as a previous image, an optical flow calculation device 60A for calculating an optical flow from a difference between the current and previous image memories 56A and 58A, and a synthetic vector calculation device 62 for calculating a motion vector by synthesizing outputs of the optical flow calculation device 60A.

During the irradiation of the charged particle beam B, the control unit 7 acquires an actual position of the tumor 14 obtained by the above-described CT scanner 40 (hereinafter, referred to as an “actually measured tumor position”). When the irradiation starts, the control unit 7 acquires the estimated tumor position included in the treatment plan map from the treatment plan device 100. Therefore, the control unit 7 can recognize the variations in the position of the tumor 14 by calculating a deviation between the actually measured tumor position and the estimated tumor position. The control unit 7 recognizes the variations in the position of the tumor 14 in the plane direction (in the XY-plane), and recognizes the variations in the position of the tumor 14 in the depth direction (in the Z-axis direction).

The control unit 7 adjusts the irradiation position of the charged particle beam B in the plane direction so as to track the variations in the position of the tumor 14 in the plane direction. That is, in a case where there is a deviation between the actually measured tumor position and the estimated tumor position in the plane direction, the control unit 7 corrects a planned irradiation position and a scanning route in accordance with the actually measured tumor position. The control unit 7 controls the irradiation unit 2 so that the irradiation using the charged particle beam B is performed in accordance with the corrected planned irradiation position and the corrected scanning route. The control unit 7 can correct the deviation of the irradiation position during alignment prior to the irradiation. Furthermore, in a case where the tumor 14 of the patient moves even after the alignment prior to the irradiation, the control unit 7 can control the irradiation position by tracking the movement. A specific processing example performed by the control unit 7 will be described later.

In a case where the position of the tumor 14 in the depth direction out of a predetermined range, the control unit 7 stops the irradiation of the charged particle beam B. If the position of the tumor 14 falls again within the predetermined range, the control unit 7 resumes the irradiation of the charged particle beam B. The control unit 7 recognizes a deviation (amount of a position deviation) between the actually measured tumor position and the estimated tumor position in the depth direction. The control unit 7 determines whether or not the deviation falls again within a predetermined threshold range. Ina case where the control unit 7 determines whether the deviation is equal to or smaller than a threshold, the control unit 7 continues the irradiation of the charged particle beam B. On the other hand, in a case where the control unit 7 determines that the deviation is greater than the threshold, the control unit 7 stops the irradiation of the charged particle beam B. A method of stopping the irradiation of the charged particle beam B is not particularly limited. For example, a method of temporarily stopping the emission of the charged particle beam B from the accelerator 3, a method of stopping a high-frequency acceleration electrode, or a method of causing a beam chopper electrode to change a trajectory of a beam emitted from an ion source so that the beam is not accelerated may be adopted. The control unit 7 continues to acquire the deviation amount, and resumes the irradiation of the charged particle beam B at a timing that the deviation amount is equal to or smaller than the threshold.

A specific processing example performed by the control unit 7 will be described later.

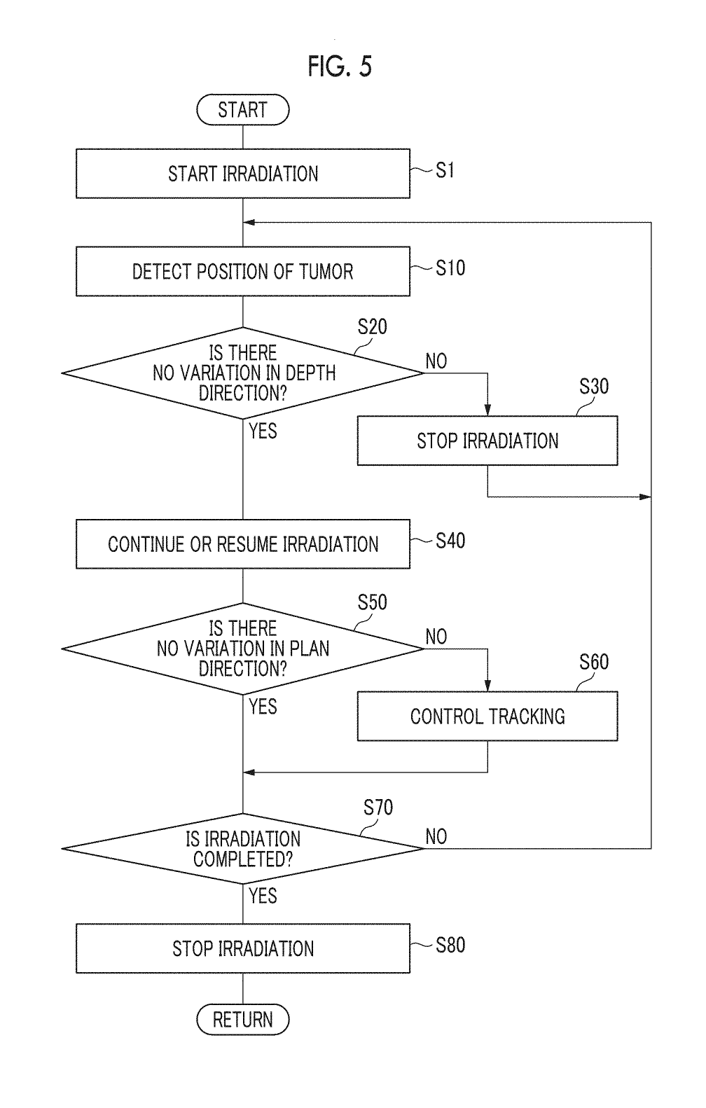

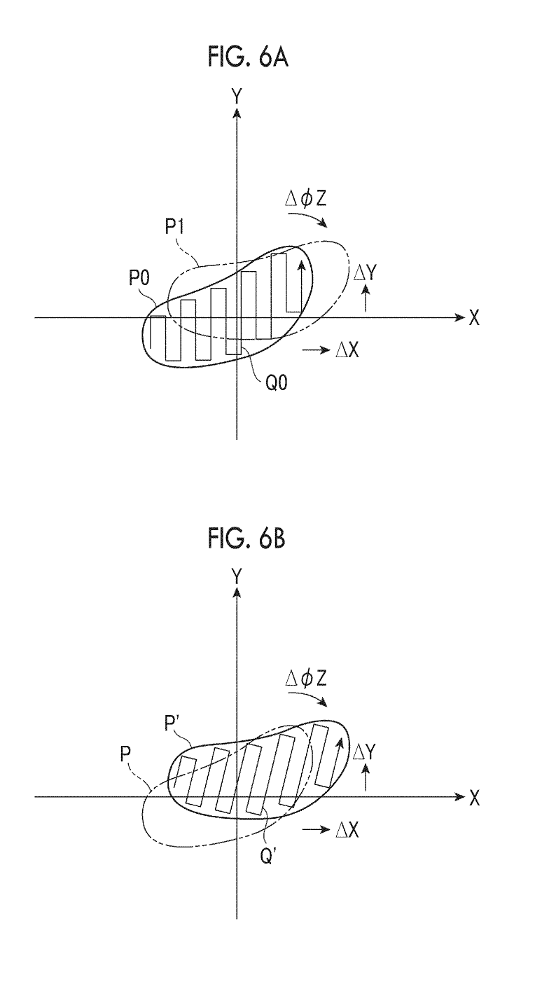

Subsequently, referring to FIGS. 5 to 6B, a procedure of the charged particle beam treatment performed by the charged particle beam treatment apparatus 1, which includes an operation of the charged particle beam treatment apparatus 1, will be described. However, content for controlling the charged particle beam treatment apparatus 1 is not limited to the following procedure.

(Irradiation Start Step: S1 in FIG. 5) First, the patient 15 is placed on the treatment table 4, and the patient 15 is aligned by using an aligning laser marker (not illustrated) of the charged particle beam treatment apparatus 1. Specifically, the treatment table 4 is moved in order to align the position of the tumor 14 of the patient 15 with the estimated tumor position determined in the treatment plan map in advance. If the alignment is completed, the control unit 7 starts irradiating the tumor 14 with the charged particle beam B.

(Lesion Area Position Detection Step: S10 in FIG. 5) After the above-described process in S1, the CT scanner 40 is driven under the control of the control unit 7, and the CT image inside the patient 15 is acquired. Based on this CT image, the control unit 7 detects the position of the tumor 14 of the patient 15. In this manner, the control unit 7 recognizes an actual position of the tumor 14 (actually measured tumor position) relative to the irradiation unit 2.

(Depth Direction Variation Determination Step: S20 in FIG. 5) Based on a detection result obtained by the CT scanner 40, the control unit 7 determines whether or not there is a variation in the position of the tumor 14 in the depth direction. If the position of the tumor 14 in the depth direction falls again within a predetermined range, the control unit 7 determines that there is no variation. If the position is out of the range, the control unit 7 determines that there is a variation. The control unit 7 reads out the treatment plan map of the treatment plan device 100, and recognizes the estimated tumor position and the scanning route which are included in the treatment plan map. The control unit 7 calculates a position deviation in the depth direction between the actually measured tumor position and the estimated tumor position. The position deviation calculated here is a deviation of a translational direction in the depth direction (Z-axis direction). The deviation of the translational direction in is called “ΔZ”. For example, with respect to the estimated tumor position, a threshold “ΔZ_{TH}” is set as an allowable value of the deviation in the Z-axis direction. In this case, if “ΔZ≤ΔZ_{TH}” is satisfied, the control unit 7 determines that there is no variation in the position of the tumor 14 in the depth direction. If “ΔZ>ΔZ_{TH}” is satisfied, the control unit 7 determines that there is a variation in the position of the tumor 14 in the depth direction.

The control unit 7 may determine any portion of the tumor 14 as reference position. For example, as illustrated in FIG. 4A, in a case where a position of a lower end SP of the tumor 14 at the estimated tumor position is set as a reference position D₀, determination positions D₁ and D₂ are set at positions separated upward and downward from the reference position D0 as much as ΔZ_{TH}. In a case where the lower end SP of the tumor 14 at the actually measured tumor position based on the detection result in S10 is located between the determination positions D₁ and D₂ or on the determination positions D₁ and D₂, the control unit 7 determines that there is no variation in the position of the tumor 14 in the depth direction. In a case where the lower end SP of the tumor 14 in the actually measured tumor position based on the detection result in S10 is located below the determination position D₁ or above the determination position D₂, the control unit 7 determines that there is a variation in the position of the tumor 14 in the depth direction. A magnitude of the threshold “ΔZ_{TH}” may be optionally set.

(Irradiation Stopping Step: S30 in FIG. 5) In S20, in a case where the control unit 7 determines that there is the variation in the position of the tumor 14 in the depth direction, the control unit 7 stops the irradiation of the charged particle beam B. Thereafter, the process returns to S10, and the same process is repeatedly performed.

(Irradiation Continuing/Resuming Step: S40 in FIG. 5) In S20, in a case where the control unit 7 determines that there is no variation in the position of the tumor 14 in the depth direction, the control unit 7 continues the irradiation of the charged particle beam B. In a case where the position of the tumor 14 falls again within a predetermined range from a state where the irradiation of the charged particle beam B is stopped in S30, the control unit 7 determines that there is no more variation (variation from the estimated tumor position serving as the reference position) in the position of the tumor 14 in the depth direction in S20. In this manner, in a case where the process proceeds to S40, the control unit 7 resumes the irradiation of the charged particle beam B.

(Plane Direction Variation Determination Step: S50 in FIG. 5) Based on the detection result in S10, the control unit 7 determines whether or not there is a variation in the position of the tumor 14 in the plane direction. For example, as illustrated in FIG. 6A, the control unit 7 reads out the treatment plan map of the treatment plan device 100, and recognizes an estimated tumor position P0 and a scanning route Q0 which are included in the treatment plan map. The control unit 7 calculates the position deviation between the actually measured tumor position P1 and the estimated tumor position P0. The position deviation calculated here is a deviation of the translational direction and a deviation of the rotation direction within a plane (XY-plane) orthogonal to the irradiation direction (Z-axis direction) of the charged particle beam B. The deviation of the above-described translational direction includes deviations in two axis directions such as a deviation in the X-axis direction and a deviation in the Y-axis direction and biaxial deviation. The former will be referred to as “ΔX”, and the latter will be referred to as “ΔY”. In addition, the deviation of the rotation direction, that is, the deviation of the rotation direction around the Z-axis will be referred to as “ΔΦZ”. The control unit 7 compares a preset threshold with the acquired ΔX, ΔY, and ΔΦZ. If all of ΔX, ΔY, and ΔΦZ are equal to or smaller than the threshold, the control unit 7 determines that there is no variation in the position of the tumor 14 in the plane direction. In S50, in a case where the control unit 7 determines that there is no variation in the position of the tumor 14 in the plane direction, the control unit 7 continues the irradiation of the charged particle beam B, based on the estimated tumor position P₀ and the scanning route Q₀, and the process proceeds to S70. If at least any one of ΔX, ΔY, and ΔΦZ exceeds the threshold, the control unit 7 determines that there is a variation in the position of the tumor 14 in the plane direction.

(Tracking Control Step: S60 in FIG. 5) In S50, in a case where the control unit 7 determines that there is a variation in the position of the tumor 14 in the plane direction, the control unit 7 adjusts the irradiation position of the charged particle beam B in the plane direction so as to track the variation in the position of the tumor 14. Based on the calculated ΔX, ΔY, and ΔΦZ, the control unit 7 corrects (converts) the planned irradiation position and the scanning route by using a correction amount which is the same as the amount of ΔX, ΔY, and ΔΦZ. That is, as illustrated in FIG. 6B, a corrected planned irradiation position P′ is totally translated as much as +ΔX and +ΔY with respect to a planned irradiation position P before correction (that is, position the same as the estimated tumor position P0 of the treatment plan map), and is located at a position rotationally moved as much as +ΔΦZ. In addition, a pattern of a corrected scanning route Q′ is totally translated as much as +ΔX and +ΔY with respect to the scanning route before correction (that is, the same route as the scanning route Q0 of the treatment plan map), and becomes a pattern rotationally moved as much as +ΔΦZ. If the process in S50 is completed, the control unit 7 performs the irradiation of the charged particle beam B, based on the corrected planned irradiation position P′ and the corrected scanning route Q′. In addition, the process proceeds to S70.

For example, after the control unit 7 performs tracking control in S60 (first time), when the process in S50 (second time) is performed, in a case where the position of the tumor 14 in the plane direction remains at the same position, in S50 (second time), the control unit 7 acquires ΔX, ΔY, and ΔΦZ which are the same as those at the first time, as the deviation between the actually measured tumor position P1 and the estimated tumor position P0. In S60 (second time), the control unit 7 acquires the planned irradiation position P′ and the scanning route Q′ which are the same as those at the first time. In this case, it seems that the irradiation position of the charged particle beam B subsequent to S60 (second time) has no apparent change when viewed from the planned irradiation position P′ and the scanning route Q′ which are set in S60 (first time). On the other hand, after the control unit 7 performs the tracking control in S60 (first time), when the process in S50 (second time) is performed, in a case where the position of the tumor 14 in the plane direction further varies, in S50 (second time), the control unit 7 acquires ΔX, ΔY, and ΔΦZ which are different from those at the first time, as the deviation between the actually measured tumor position P1 and the estimated tumor position P0. In S60 (second time), the control unit 7 acquires the planned irradiation position P′ and the scanning route Q′ which are different from those at the first time. In this case, the irradiation position of the charged particle beam B subsequent to S60 (second time) varies from the planned irradiation position P′ and the scanning route Q′ which are set in S60 (first time). After the control unit 7 performs the tracking control in S60 (first time), when the process in S50 (second time) is performed, in a case where the position of the tumor 14 in the plane direction returns to the estimated tumor position P0, the control unit 7 performs the irradiation of the charged particle beam B, based on the estimated tumor position P0 and the scanning route Q0. In this case, the irradiation position of the charged particle beam B subsequent to S50 (second time) varies from the planned irradiation position P′ and the scanning route Q′ which are set in S60 (first time).

(Irradiation Completion Determination Step: S70 in FIG. 5) The control unit 7 determines whether or not the tumor 14 is completely irradiated with the charged particle beam B. In a case where all of the layers L₁ to L_{N} are irradiated with the charged particle beam B, the control unit 7 determines that the irradiation is completed. In S70, in a case where the control unit 7 determines that the irradiation of the charged particle beam B is not completed, the process returns to S10, and the same process is repeatedly performed.

(Irradiation Stopping Step: S80 in FIG. 5) In S70, in a case where the control unit 7 determines that the tumor 14 is completely irradiated with the charged particle beam B, the control unit 7 stops the irradiation of the charged particle beam B. In this manner, the process illustrated in FIG. 5 is completed.

For example, the control unit 7 is physically configured to serve as a computer system including a CPU, a RAM, a ROM, an auxiliary storage device, an input device such as a keyboard and a mouse, an output device such as a display, and a communication module. Then, a predetermined irradiation control program is executed in the computer system serving as the control unit 7. In this way, the charged particle beam treatment apparatus 1 is operated so as to perform S1 to S80 as described above. The above described steps S1 to S80 may be automatically performed under the control of the control unit 7, or may be performed in a batch processing manner in accordance with an operation of an operator.

How frequently the position detection process of the tumor 14 in S10 is performed (that is, how frequently the process in S10 to S70 is performed) is not particularly limited. For example, during the irradiation of the charged particle beam B, the control unit 7 may always repeat the process in S10 to S70. Alternatively, the control unit 7 may repeat the process in S10 to S70, based on a predetermined time interval. Alternatively, the control unit 7 may perform the process in S10 to S70 at a timing that the layer L serving as the irradiation target is switched. An operator may operate a switch so that the control unit can determine whether or not to stop the irradiation.

Next, an operation and an advantageous effect of the charged particle beam treatment apparatus 1 according to the present embodiment will be described.

The irradiation position of the charged particle beam in the plane direction can be promptly adjusted. That is, in order to adjust the depth direction, the beam needs to pass through a device for converting energy. However, this device takes time to operate an energy conversion mechanism. On the other hand, the plane direction can be adjusted only by applying electrical correction to the scanning electromagnet. Accordingly, the irradiation position can be promptly adjusted. Therefore, the irradiation position of the charged particle beam B in the plane direction can be promptly adjusted by inputting a signal from the lesion area position detection unit 50 to the control unit 7 on a real-time basis so that the control unit 7 corrects the irradiation axis. Therefore, the control unit 7 adjusts the irradiation position of the charged particle beam B in the plane direction so as to track the variation in the position of the tumor 14 in the plane direction. In this manner, the irradiation position of the charged particle beam B can satisfactorily track the variation in the position of the tumor 14 in the plane direction. On the other hand, in order to adjust the irradiation position of the charged particle beam B in the depth direction, it is necessary to change the energy of the charged particle beam B. Accordingly, it requires time to adjust the irradiation position of the charged particle beam B. Therefore, in a case where the position of the tumor 14 in the depth direction is out of a predetermined range, the control unit 7 stops the irradiation of the charged particle beam B. If the position of the tumor 14 falls again within the predetermined range, the control unit 7 resumes the irradiation of the charged particle beam B. That is, the control unit 7 performs the irradiation of the charged particle beam B, when the position of the tumor 14 in the depth direction less deviates and a planned location can be irradiated with the charged particle beam B can be irradiated to the scheduled place. When the position of the tumor 14 in the depth direction greatly deviates, the control unit 7 stops the irradiation so that an unplanned location is not irradiated with the charged particle beam B. In this manner, the irradiation unit 2 can prevent the unplanned location from being irradiated with the charged particle beam B. According to the above-described configuration, it is possible to improve accuracy in irradiating the tumor 14 with the charged particle beam B.

The lesion area position detection unit 50 includes the CT scanner 40 which acquires the CT image of the tumor 14. In this case, the lesion area position detection unit 50 can accurately detect the position of the tumor 14.

The present invention including the above-described embodiment can be embodied in various forms to which various modifications and improvements are added based on the knowledge of those skilled in the art. In addition, the following modification examples can be configured by utilizing the technical ideas described above in the embodiment. The configurations of the respective embodiments maybe appropriately combined with each other.

For example, in the above-described embodiment, the lesion area position detection unit 50 includes the CT scanner 40. Alternatively, the lesion area position detection unit 50 may detect the position of the lesion area by detecting a body surface motion of the patient 15 and estimating the position of the lesion area from the body surface motion. The lesion area position detection unit 50 can detect the position of the lesion area without using a large-scale device such as the CT scanner 40. In this case, the lesion area position detection unit 50 may include a camera for imaging the body surface of the patient 15.

In the above-described embodiment, after the control unit 7 performs the tracking control in S60 (first time), when the process in S50 (second time) is performed, the control unit 7 calculates the deviation between the actually measured tumor position P1 and the estimated tumor position P0. Alternatively, when the control unit 7 performs the process in S50 (second time), the control unit 7 may calculate the deviation between the actually measured tumor position P1 and the planned irradiation position P′ which is corrected in S60 (first time). In this case, in a case where the tumor 14 is not moved from the position in the process in S60 (first time), the control unit 7 can omit the calculation in S60 (second time).

In the above embodiment, the irradiation of the corrected planned irradiation position is realized by the scanning of the scanning electromagnet 6 in the scanning method. However, the present invention is not limited to this method. For example, the present invention may be applied to a broad beam irradiation method, that is, a method in which the irradiation field of the charged particle beam B is defined by an opening state of the multi-leaf collimator 24 (FIG. 2). In this case, in the irradiation control step S109, the position (for example, position of X, Y, and ΦZ) of the opening portion 24c of the multi-leaf collimator 24 may be controlled by the control unit 7 so as to correspond to the corrected planned irradiation position P′.

If a method of using a patient collimator is used, in the irradiation control step S109, the position of the patient collimator may be controlled instead of the control of the multi-leaf collimator 24. That is, the position (for example, position of X, Y, and ΦZ) of the patient collimator (for example, X, Y, ΦZ position) may be controlled by the control unit 7 so as to correspond to the corrected planned irradiation position P′. In this case, an actuator for moving the position of the patient collimator may be disposed so that the actuator is controlled by the control unit 7.

The scanning of the scanning electromagnet 6 and the position of the opening portion 24c of the multi-leaf collimator 24 as described above may be controlled in parallel with each other. The charged particle beam treatment apparatus 1 according to the embodiment includes both the scanning electromagnet 6 and the multi-leaf collimator 24. However, it is not indispensable to provide both the configuration elements, and the configuration elements may be appropriately omitted.

It shouldbe understood that the invention is not limited to the above-described embodiment, but may be modified into various forms on the basis of the spirit of the invention. Additionally, the modifications are included in the scope of the invention.

What is claimed is:

1. A charged particle beam treatment apparatus comprising:
an irradiation unit that irradiates a patient with a charged particle beam;
a lesion area position detection unit that detects a position of a lesion area of the patient; and
a control unit that controls the irradiation unit, based on the position of the lesion area detected by the lesion area position detection unit,
wherein the control unit adjusts an irradiation position of the charged particle beam in a plane direction orthogonal to an irradiation axis of the charged particle beam so as to track variations in the position of the lesion area in the plane direction, and
wherein in a case where the position of the lesion area in a depth direction along the irradiation axis is out of a predetermined range, the control unit stops irradiation using the charged particle beam, and in a case where the position of the lesion area falls again within the predetermined range, the control unit resumes the irradiation using the charged particle beam.

2. The charged particle beam treatment apparatus according to claim 1,
wherein the lesion area position detection unit includes a CT scanner which acquires a CT image of the lesion area.

3. The charged particle beam treatment apparatus according to claim 1,
wherein the lesion area position detection unit detects a body surface motion of the patient, and detects the position of the lesion area by estimating the position of the lesion area from the body surface motion.
